(19) United States
(12) Patent Application Publication
(10) Pub. No.: US 2023/0257714 A1
(43) Pub. Date: Aug. 17, 2023

(54) METHODS AND COMPOSITIONS FOR GENERATING HUMAN INDUCED MESENCHYMAL STEM CELLS

(71) Applicant: SmartCella Solutions AB, Tullinge (SE)

(72) Inventors: Ran YANG, Solna (SE); Kenneth R. CHIEN, Cambridge, MA (US)

(21) Appl. No.: 18/106,327

(22) Filed: Feb. 6, 2023

Related U.S. Application Data
(60) Provisional application No. 63/307,368, filed on Feb. 7, 2022.

(51) Int. Cl. C12N 5/0775

(52) U.S. Cl. CPC C12N 5/0668; C12N 2501/415; C12N 2501/105; C12N 2501/115; C12N 2501/135; C12N 2500/32; C12N 2506/02

(57) ABSTRACT

Methods for generating human induced mesenchymal stem cells (iMSC) from human pluripotent stem cells, such as embryonic stem cells, are provided. Progenitors of iMSCs are first generated in a two-step protocol, with further differentiation to iMSCs accomplished by a third step culture. The iMSCs express mesenchymal surface markers and exhibit trilineage differentiation to adipocytes, osteocytes and chondrocytes. Culture media, methods of isolating extracellular vesicles from the iMSCs and kits are also provided.

### RELATED APPLICATIONS

This application claims priority to U.S. Provisional Application No. 63/307,368, filed Feb. 7, 2022. The entire contents of the prior application are hereby incorporated by reference in their entirety.

### BACKGROUND OF THE INVENTION

Mesenchymal stem cells (MSCs) are non-hematopoietic adult stem cells that have the ability to self-renew and also exhibit multilineage differentiation. MSCs can be isolated from a variety of tissues, such as umbilical cord, bone marrow and adipose tissue, as well as amniotic fluid, menstrual blood and endometrium. MSCs are multipotent stromal cells that can differentiate into a variety of cell types, including osteocytes, chondrocytes, myocytes and adipocytes. MSCs also have been shown to have immunomodulatory effects, including the ability to produce immunomodulatory molecules, such as cytokines. The multipotent properties of MSCs make them an attractive choice for possible development of clinical applications.

There are a number of limitations, however, to vigorous in vitro expansion of ex vivo isolated adult MSCs, including a decline of plasticity and potency over time, as well as accumulation of DNA abnormalities and replicative senescence (see e.g., Rombouts and Ploemacher (2003) Leukemia 17:160-170; Miura et al. (2006) Stem Cells 24:1095-1103; Kyriakou et al. (2008) Haematologica 93:1457-1465; Liu et al. (2012) PLoS One 7:e33225). Additionally, variation in quality of cells across donors and tissue sources has led to inconsistencies in the reported in vivo effectiveness of MSCs (see e.g., Wagner and Ho (2007) Stem Cell Rev. 3:239-248; Galipeau (2013) Cytotherapy 15:2-8; Kimbrel (2014) Stem Cells and Develop. 23:1611-1624; Tyndall (2014) Nat. Rev. Rheumatol. 10:117-124). Thus, there still is a need in the art for more reliable and consistent sources of MSCs.

Attempts have been made to generate MSCs from other stem cells in vitro. For example, approaches for generating MSCs from human induced pluripotent stem cells (iPSCs) have been described (see e.g., Hynes et al. (2014) Stem Cells. Dev. 23:1084-1096; Yang et al. (2014) PLoS One 9:e100285; Kang et al. (2015) Stem Cell Res. Ther. 6:144; Lin et al. (2016) Curr. Stem Cell Res. Ther. 11:122-130; Spitzhorn et al. (2018) Stem Cells Dev. 27:1702-1714; Wang et al. (2018) Stem Cells 36:903-914; Yang et al. (2019) Cell Death & Disease 10:718; Xu et al. (2019) Stem Cells 37:754-765; Spitzhorn et al. (2019) Stem Cell Res. Ther. 10:100). Such cells are referred to in the art as iPSC-MSCs.

While some progress has been, there remains a need for efficient and robust methods and compositions for generating human induced mesenchymal stem cells in culture from human pluripotent stem cells.

### SUMMARY OF THE INVENTION

This disclosure provides methods for generating human induced mesenchymal stem cells (iMSCs), and progenitor cells thereof, from human pluripotent stem cells (e.g., human embryonic stem cells) using a three-step protocol that generates iMSCs in as little as 21 days of culture. The disclosure first provides a two-step protocol that allows for obtention of iMSC progenitor cells in as little as fourteen days of culture. A third step, with an additional culture of at least seven days or more, allows for the obtention of mature iMSCs that express mesenchymal surface markers and that have the capacity for trilineage differentiation to adipocytes, osteocytes and chondrocytes upon further culture under appropriate cell-specific differentiation conditions. The iMSCs also exhibit immunomodulatory capabilities. The methods of the disclosure can be used to obtain iMSC progenitor cells, as well as mature iMSCs and further differentiated cells obtainable from iMSCs.

Accordingly, in one aspect, the disclosure pertains to a method of generating human mesenchymal stem cell progenitor cells comprising:

(a) culturing human pluripotent stem cells in a culture media comprising a WNT pathway agonist and a BET pathway antagonist for at least two days (or at least three days or for four days), such as on day 0 to day 4 to generate induced cells; and

(b) culturing the induced cells from step (a) in a culture media comprising a PDGF pathway agonist, an IGF1 pathway agonist and an FGF-beta pathway agonist for at least 10 days (or at least 11, 12, 13 or 14 days), such as on day 4 to day 14 to generate human mesenchymal stem cell progenitor cells.

In embodiments, the human pluripotent stem cells are human embryonic stem cells.

In embodiments, the WNT pathway agonist is a glycogen synthase kinase 3 (Gsk3) inhibitor, such as CHIR98014. In embodiments, the WNT pathway agonist is selected from the group consisting of CHIR98014, CHIR99021, SB 216763, SB 415286, LY2090314, 3F8, A 1070722, AR-A 014418, BIO, AZD1080, WNT3A, and combinations thereof. In embodiments, the WNT pathway agonist is present in the culture media at a concentration within a range of 0.25-0.75 μM. In embodiments, the WNT pathway agonist is present in the culture media at a concentration of 0.5 M. In an embodiment, the WNT pathway agonist is CHIR98014 at a concentration of 0.5 μM.

In embodiments, the BET pathway antagonist is a triazolo-diazepine compound, such as (+)-JQ1. In embodiments, the BET pathway antagonist is selected from the group consisting of (+)-JQ1, TEN-010, OTX015, I-BET762, I-BET151, BAY1238097, ABBV-744, ABBV-075, iBET-BD1, iBET-BD2, SJ432, RVX-208, MS417, AZD5153, and combinations thereof. In embodiments, the BET pathway antagonist is present in the culture media at a concentration within a range of 25-75 nM. In an embodiment, the BET pathway antagonist is (+)-JQ1 at a concentration of 50 nM.

In embodiments, the PDGF pathway agonist is PDGF-BB. In embodiments, PDGF-BB is present in the culture media at a concentration within a range of 7.5-12.5 ng/ml. In an embodiment, the PDGF pathway agonist is PDGF-BB, which is present in the culture media at a concentration of 10 ng/ml.

In embodiments, the IGF1 pathway agonist is IGF1. In embodiments, IGF1 is present in the culture media at a concentration within a range of 15-25 ng/ml. In an embodiment, the IGF1 pathway agonist is IGF1, which is present in the culture media at a concentration of 20 ng/ml.

In embodiments, the FGF-beta pathway agonist is FGF-beta. In embodiments, FGF-beta is present in the culture media at a concentration within a range of 7.5-12.5 ng/ml. In an embodiment, the FGF-beta pathway agonist is FGF-beta, which is present in the culture media at a concentration of 10 ng/ml.

In embodiments, the culture media in step (a) and step (b) of the method comprises a base media comprising serum. In an embodiment, the base media comprising serum is DMEM/F12 media with 10-15% fetal bovine serum.

In an embodiment, the method further comprises culturing the human mesenchymal stem cell progenitor cells for at least 7 days (e.g., 14 days or more) in a culture medium comprising a base media comprising an L-glutamine supplement. In an embodiment, the L-glutamine supplement is an L-alanine-L-glutamine dipeptide. In an embodiment, the base media comprising an L-glutamine supplement is DMEM/F12 media with 10% fetal bovine serum supplemented with GlutaMAX™.

In another aspect, the disclosure pertains to a method of generating human induced mesenchymal stem cells (iMSC) comprising:

(a) culturing human pluripotent stem cells in a culture media comprising a WNT pathway agonist and a BET pathway antagonist for at least two days (or at least three days or for four days), such as on day 0 to day 4 to generate induced cells;

(b) culturing the induced cells from step (a) in a culture media comprising a PDGF pathway agonist, an IGF1 pathway agonist and an FGF-beta pathway agonist for at least 10 days (or at least 11, 12, 13 or 14 days), such as on day 4 to day 14 to generate human mesenchymal stem cell progenitor cells; and

(c) culturing the human mesenchymal stem cell progenitor cells from step (b) in a culture media comprising a base media and an L-glutamine supplement for at least 7 days to generate iMSCs.

In embodiments, step (c) comprises culturing the human mesenchymal stem cell progenitor cells in the base media and L-glutamine supplement for longer than 7 days, e.g., for at least 14 days, at least 21 days, or at least 28 days or more.

In embodiments, the human pluripotent stem cells are human embryonic stem cells.

In embodiments, the iMSCs generated in step (c) express one or more surface markers selected from the group consisting of CD73, CD90, CD105, CD29, CD44, and combinations thereof.

In embodiments, the iMSCs generated in step (c) are capable of further differentiation into adipocytes, osteocytes or chondrocytes.

Suitable WNT pathway agonists, BET pathway antagonists, PDGF pathway agonists, IGF1 pathway agonists and FGF-beta pathway agonists, and concentrations thereof, are described above and further herein.

In embodiments, the culture media in steps (a), (b) and (c) comprises a base media comprising serum. In embodiments, the base media comprising serum is DMEM/F12 media with 10-15% fetal bovine serum.

In embodiments, the L-glutamine supplement used in step (c) is an L-alanine-L-glutamine dipeptide. In an embodiment, the base media comprising an L-glutamine supplement in step (c) is DMEM/F12 media with 10% fetal bovine serum supplemented with GlutaMAX™.

In yet another aspect, the method comprises, after step (c), isolating from the culture extracellular vesicles generated by the iMSCs. Additionally, or alternatively, extracellular vesicles generated by the iMSC progenitors can be isolated after step (b).

In yet another aspect, the disclosure pertains to a two-stage culture media for obtaining human mesenchymal stem cell progenitor cells, the two-stage culture media comprising (i) a first stage media comprising a WNT pathway agonist and a BET pathway antagonist; and (ii) a second stage media comprising a PDGF pathway agonist, an IGF1 pathway agonist and an FGF-beta pathway agonist. Suitable WNT pathway agonists, BET pathway antagonists, PDGF pathway agonists, IGF1 pathway agonists and FGF-beta pathway agonists, and concentrations thereof, are described above and further herein.

Other features and advantages of the invention will be apparent from the following detailed description and claims.

### BRIEF DESCRIPTION OF THE DRAWINGS

FIG. 1 is a schematic diagram of a representative three-stage iMSC differentiation protocol of the disclosure.

FIG. 2A-B are photographs showing morphology of cells during the iMSC differentiation protocol. FIG. 2A (left and right panels) shows cells at the mesodermal stage. FIG. 2B shows cells at the mesenchymal stage at low density (10×)(left panel) or high density (5×)(right panel).

FIG. 3A-B are graphs showing the results of FACS analysis for positive MSC markers (FIG. 3A) and negative MSC markers (FIG. 3B). Markers tested are shown above graphs. The negative cocktail contained anti-CD11b, anti-CD19, anti-CD34, anti-CD45 and anti-HLA-DR antibodies.

FIG. 4A-C are photographs showing morphology of iMSCs following differentiation to adipocytes (FIG. 4A), osteocytes (FIG. 4B) or chondrocytes (FIG. 4C).

FIG. 5 is a bar graph showing increased expression of immunomodulatory genes in iMSCs by treatment with immune stimulators (ds-RNA or IFNr).

FIG. 6 are graphs showing the results of mixed lymphocyte reactions using hPBMCs only (left panel), hPBMCs+inactivated iMSCs (middle panel) or hPBMCs+activated MSCs (right panel). Results show hPBMC proliferation.

FIG. 7 is a bar graph showing results of an ELISA test for IL-10 secretion by iMSCs treated with low or high dose IFNr.

FIG. 8 are graphs showing the results of FACS-based analysis targeting Tetraspanin protein to quantify extracellular vesicle production by iMSCs.

### DETAILED DESCRIPTION OF THE INVENTION

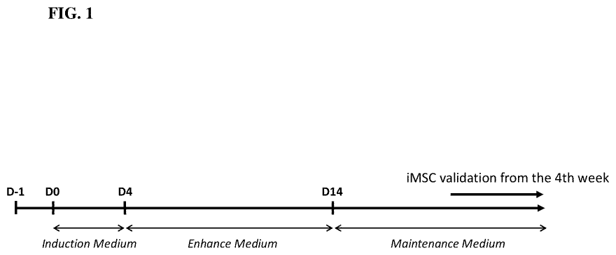

Described herein are methodologies and compositions that allow for generation of human induced mesenchymal stem cells (iMSCs) under defined culture conditions using a multi-step protocol that first generates iMSC progenitors, which then can be further differentiated into mature iMSCs. As described in Example 1, a three-step protocol, illustrated schematically in FIG. 1, has been developed for generating iMSCs from human pluripotent stem cells (e.g., human embryonic stem cells). As described in Example 2, the iMSCs express mesenchymal surface markers and lack expression of pluripotent markers and hematopoietic markers. As described in Example 3, the iMSCs are capable of trilineage differentiation, maturing into adipocytes, osteocytes or chondrocytes when cultured under appropriate cell-specific differentiation conditions. As described in Example 4, the iMSCs exhibit immunomodulatory capacities, including upregulation of immunomodulatory genes when treated with immune stimulators, the ability to modulate mixed lymphocyte reactions and the ability to secrete an anti-inflammatory factor upon stimulation with interferon-gamma (IFNr). Furthermore, as described in Example 5, the iMSCs, and progenitors thereof, as described herein are a source for extracellular vesicles (EVs), such as exosomes, that can be harvested from the cell cultures.

Various aspects of the invention are described in further detail in the following subsections.

### I. Cells

The starting cells used in the cultures of the disclosure are human pluripotent stem cells. As used herein, the term “human pluripotent stem cell” (abbreviated as hPSC) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can also evidenced by the expression of embryonic stem (ES) cell markers, although the preferred test for pluripotency is the demonstration of the capacity to differentiate into cells of each of the three germ layers.

Human pluripotent stem cells include, for example, human embryonic stem cells, such as ES cell lines, and induced pluripotent stem cells (iPSC). Non-limiting examples of human embryonic stem cell lines include ES03 cells (WiCell Research Institute) and H9 cells (Thomson, J. A. et al. (1998) Science 282:1145-1147), as well as hypo-immune embryonic stem cells including hESC SKO-CIITA, SKO-B2M or DKO (Petrus-Reurer et al. (2020) Stem Cell Reports 14:648-662). Non-limiting examples of induced pluripotent stem cells (iPSC) include 19-11-1, 19-9-7 or 6-9-9 cells (e.g., as described in Yu, J. et al. (2009) Science 324:797-801), Foreskin (clone 1 to clone 4) and IMR90 (clone 1 to clone 4) (Yu et al. (2007) Science 318:1917-20). Human pluripotent stem cells (PSCs) express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. In embodiments, the mature iMSCs generated by the methods of the disclosure lack expression of the pluripotent stem cell marker TRA-1-60.

The pluripotent stem cells are subjected to culture conditions, as described herein, that induce cellular differentiation. As used herein, the term “differentiation” refers to the development of a cell from a more primitive stage towards a more mature (i.e., less primitive) cell, typically exhibiting phenotypic features of commitment to a particular cellular lineage. In the iMSC differentiation methods described herein, progenitors of iMSCs are first generated, followed by further differentiation into mature iMSCs.

As described in Example 2, the mature iMSCs generated by the differentiation methods express mesenchymal surface markers, such as CD73, CD90, CD105, CD29 and/or CD44. In embodiments, the iMSCs express one or more mesenchymal surface markers selected from the group consisting of CD73, CD90, CD105, CD29, CD44, and combinations thereof. In embodiments, in a population of iMSCs at least 90% (or at least 95%, or at least 98% or at least 99%) of live cells express one or more mesenchymal surface markers selected from the group consisting of CD73, CD90, CD105, CD29, CD44, and combinations thereof.

Also as described in Example 2, the mature iMSCs generated by the differentiation methods lack expression of at least one pluripotent stem cell marker (e.g., TRA-1-60) and/or at least one hematopoietic cell marker (e.g., CD34, CD45 and/or HLA-DR). In embodiments, the mature iMSCs lack expression of one or more markers selected from the group consisting of TRA-1-60, CD11b, CD19, CD34, CD45, HLA-DR, and combinations thereof. In embodiments, in a population of iMSCs less 5% (or less than 3% or less than 2% or less than 1%) of live cells express one or more surface markers selected from the group consisting of TRA-1-60, CD11b, CD19, CD34, CD45, HLA-DR, and combinations thereof.

### II. Culture Media Components

The method of the disclosure for generating human induced mesenchymal stem cells comprise culturing human pluripotent stem cells (e.g., human ESCs) in culture media comprising specific agonist and/or antagonists of cellular receptors and/or signaling pathways.

In the first step (referred to herein as step (a)) of the multi-step protocol, illustrated schematically in FIG. 1, human pluripotent stem cells are cultured for at least two days, or at least three days or for four days (day 0 to day 4) in an induction medium that comprises a WNT pathway agonist and a BET pathway antagonist. In the second step (referred to herein as step (b)), the induced cells from step (a) are further cultured for at least ten days (or at least 11, 12, 13, or 14 days), such as on day 4 to day 14 (following a 4-day induction step) in an enhancement medium comprising a PDGF pathway agonist, an IGF1 pathway agonist and an FGF-beta pathway agonist, which generates iMSC progenitor cells. Finally, in the third step (referred to herein as step (c)), the iMSC progenitor cells from step (b) are further cultured for at least 7 days or more in a maintenance medium comprising an L-glutamine supplement to generate mature iMSCs.

As used herein, an “agonist” of a cellular receptor or signaling pathway is intended to refer to an agent that stimulates (upregulates) the cellular receptor or signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

Agonists of the WNT pathway include agents, molecules, compounds, or substances capable of stimulating (upregulating) the canonical Wnt/β-catenin signaling pathway, which biologically is activated by binding of a Wnt-protein ligand to a Frizzled family receptor. In one embodiment, a WNT pathway agonist is a glycogen synthase kinase 3 (Gsk3) inhibitor, such as CHIR98014. In one embodiment, the WNT pathway agonist is selected from the group consisting of CHIR98014, CHIR99021, SB 216763, SB 415286, LY2090314, 3F8, A 1070722, AR-A 014418, BIO, AZD1080, WNT3A, and combinations thereof. In one embodiment, the WNT pathway agonist is present in the culture media at a concentration within a range of 0.25-0.75 μM, 0.3-0.7 μM, 0.4-0.6 μM or 0.45-0.55 μM. In one embodiment, the WNT pathway agonist is present in the culture media at a concentration of 0.5 μM. In one embodiment, the WNT pathway agonist is CHIR98014. In one embodiment, the WNT pathway agonist is CHIR98014, which is present in the culture media at a concentration within a range of 0.25-0.75 μM, 0.3-0.7 μM, 0.4-0.6 μM or 0.45-0.55 μM. In one embodiment, the WNT pathway agonist is CHIR98014, which is present in the culture media at a concentration of 0.5 μM.

Antagonists of the BET pathway include agents, molecules, compounds, or substances capable of inhibiting (downregulating) BET proteins (“bromodomain and extra-terminal motif” proteins), which comprise a bromodomain(s). Bromodomains are protein interaction modules that selectively recognize e-N-acetylated lysine residues (Kac). The human BET family (BRD2, BRD3, BRD4 and BRDT), which all contain two conserved bromodomains per target, plays a key role regulating transcription of growth stimulating genes.

In an embodiment, the BET pathway antagonist is a triazolo-diazepine compound, non-limiting examples of which include (+)-JQ1 (described in, e.g., Filippakopoulos et al. (2010) Nature 468:1067-1073), TEN-010 (described in, e.g., Finley and Copeland (2014) Chem. Biol. 21:1196-1210), OTX015 (described in, e.g., Seal et al. (2012) Bioorg. Med. Chem. Lett. 22:2968-2972), as well as structurally-related compounds. In an embodiment, the triazolo-diazepine compound is (+)-JQ1 (Sigma Aldrich, Cat. #SML1524). Various other BET pathway antagonists have been described (reviewed in, e.g., Cochran et al. (2019) Nat. Rev. Drug Disc. 18:609-628; and Zaware et al. (2019) Nat. Struct. Mol. Biol. 26:870-879). In embodiments, the BET pathway antagonist is selected from the group consisting of (+)-JQ1, TEN-010, OTX015, I-BET762, I-BET151, BAY1238097, ABBV-744, ABBV-075, iBET-BD1, iBET-BD2, SJ432, RVX-208, MS417, AZD5153, and combinations thereof. In embodiments, the BET pathway antagonist is present in the culture media at a concentration within a range of 25-75 nM, 30-70 nM, 40-60 nM or 45-55 nM. In an embodiment, the BET pathway antagonist is present in the culture media at a concentration of 50 nM. In embodiments, the BET pathway antagonist is (+)-JQ1, which is present in the culture media at a concentration within a range of 25-75 nM, 30-70 nM, 40-60 nM or 45-55 nM. In an embodiment, the BET pathway antagonist is (+)-JQ1 at a concentration of 50 nM.

Agonists of the PDGF (platelet-derived growth factor) pathway include agents, molecules, compounds, or substances capable of stimulating (upregulating) a signaling pathway initiated by binding of a PDGF (e.g., PDGF-AA, PDGF-AB or PDGF-BB) to its receptor. In an embodiment, the PDGF pathway agonist is PDGF-BB (e.g., R&D Systems, Cat. #220-BB). In an embodiment, the PDGF agonist is 740Y-P (Tocris, Cat. #1983). In an embodiment, the PDGF pathway agonist is PDGF-BB, which is present in the culture media at a concentration within a range of 7.5-12.5 ng/ml, 8.0-12.0 ng/ml, 9.0-11.0 ng/ml, 9.5-10.5 ng/ml or 10 ng/ml.

Agonists of the IGF1 (insulin-like growth factor 1) pathway include agents, molecules, compounds, or substances capable of stimulating (upregulating) a signaling pathway initiated by binding of IGF1 to its receptor. In an embodiment, the IGF1 pathway agonist is IGF1 (e.g., R&D Systems, Cat. #291-G1). Other examples of IGF1 pathway agonists include agonistic peptides, such as IGF1 30-41 peptide and IGF1 24-41 peptide. In an embodiment, the IGF1 pathway agonist is IGF1, which is present in the culture media at a concentration within a range of 15-25 ng/ml, 16.5-23.5 ng/ml, 17.5-22.0 ng/ml, 19.0-21.0 ng/ml or 20 ng/ml.

Agonists of the FGF-beta (fibroblast growth factor-beta) pathway include agents, molecules, compounds, or substances capable of stimulating (upregulating) a signaling pathway initiated by binding of FGF-beta to its receptor. In an embodiment, the FGF-beta pathway agonist is FGF-beta (e.g., R&D Systems, Cat. #3718-FB). In an embodiment, the FGF-beta pathway agonist is FGF-beta, which is present in the culture media at a concentration within a range of 7.5-12.5 ng/ml, 8.0-12.0 ng/ml, 9.0-11.0 ng/ml, 9.5-10.5 ng/ml or 10 ng/ml.

In embodiments, the culture media in step (a) and step (b) of the iMSC differentiation protocol comprises a base media. In an embodiment, the base media is DMEM/F12 media, although other media of similar components to DMEM/F12 are also suitable for use as base media. In embodiments, the base media comprises serum. In embodiments, the serum is selected from fetal bovine serum and human serum. In an embodiment, the base media (in step (a), in step (b), or in both steps (a) and (b)) is DMEM/F12 media with 10% fetal bovine serum.

The iMSC progenitor cells generated by steps (a) and (b) of the differentiation protocol are further cultured in a third step (referred to herein as step (c)) to generate mature iMSCs. This step comprises culturing the human mesenchymal stem cell progenitor cells from step (b) in a culture media comprising a base media and an L-glutamine supplement for at least 7 days to generate iMSCs. In embodiments, the progenitor cells are cultured for at least 14 days, at least 21 days, at least 28 days, at least one month or at least two months, to generate iMSCs.

In embodiments, the L-glutamine supplement is an L-alanine-L-glutamine dipeptide. In an embodiment, the L-alanine-L-glutamine dipeptide is GlutaMAX™ (e.g., Thermo Fisher, Cat. #35050-061). In an embodiment, the L-glutamine supplement is L-glutamine (e.g., Thermo Fisher, Cat. #25030081).

In an embodiment, the base media is DMEM/F12 media, although other media of similar components to DMEM/F12 are also suitable for use as base media. In embodiments, the base media comprises serum. In embodiments, the serum is selected from fetal bovine serum and human serum. In an embodiment, the base media in step (c) is DMEM/F12 media with 10-15% fetal bovine serum (e.g., 10% FBS). In an embodiment, the culture media used in step (c) comprises DMEM/F12 media with 10-15% fetal bovine serum (e.g., 10% FBS) supplemented with GlutaMAX™.

### III. Culture Conditions

In combination with the defined culture media described in subsection II above, the methods of generating iMSCs of the disclosure utilize standard culture conditions established in the art for cell culture. For example, cells can be cultured at 37° C. and under 20% O₂ and 5% CO₂ conditions. A base media can be used as the starting media to which supplemental agents can be added. For example, in an embodiment, a commercially available DMEM/F12 media containing L-glutamine (Thermo Fisher, Cat. #11320033) can be used as the base media. Alternatively, DMEM/F12 media without L-glutamine (Thermo Fisher, Cat. #21331046) can be used as the base media and supplemented with L-glutamine or with a stabilized form of a glutamine supplement, such as GlutaMAX™. Other examples of similar base media include Advanced DMEM/F12 media (Thermo Fisher, Cat. #12364-010), HPLM media (Thermo Fisher, Cat. #A4899101) and aMEM media (Thermo Fisher, Cat. #22571020).

Other media suitable for use as base media for culture of human pluripotent stem cells that are known in the art include, but are not limited to, mTeSR1 (STEMCELL Technologies), Essential 8™ Medium (Thermo Fisher), PeproGrow™ hESC Basal Media (PeproTech) and StemFlex Medium (Thermo Fisher). Cells can be cultured in standard culture vessels or plates, such as culture dishes, culture flasks or 96-well plates. The starting human pluripotent cells can be obtained as cell lines well established in the art (e.g., human ESCs or iPSCs).

In various embodiments of the methods of the disclosure, the cells resulting from the three-step protocol described in Example 1 are cultured for a sufficient time such that at least 90% (more preferably at least 91%, 92%, 93%, 94%, 95%, 96%, 97%, 98% or 99%) of the cells within the cell population express at least one, and preferably multiple (e.g., at least two, three, four or five) mesenchymal cell surface markers. Non-limiting examples of mesenchymal cell surface markers include CD73, CD90, CD105, CD29, CD44, and combinations thereof. In embodiments, the cells resulting from the three-step protocol described in Example 1 are cultured for a sufficient time such that less than 5% (more preferably less than 4%, 3%, 2%, 1%, or 0.5%) of the cells within the cell population express one or more markers selected from the group consisting of TRA-1-60, CD34, CD45, HLA-DR, and combinations thereof.

In embodiments, the iMSCs generated by the protocol described in Example 1 can be further treated for immunomodulatory effects (see Example 4). For example, to upregulate immunomodulatory genes in the iMSCs, the cells can be treated with an immune stimulator, such as double-strand RNA (ds-RNA) or interferon-gamma (IFN-r). The iMSCs can be stimulated to secrete anti-inflammatory factors, such as interleukin-10 (IL-10) by treatment with IFN-r.

In embodiments, the iMSCs generated by the protocol described in Example 1 can be subjected to further differentiation under conditions to generate specific cell types, including but not limited to adipocytes, osteocytes or chondrocytes. Conditions for generating such cells from iMSCs are well established in the art (see e.g., Example 3).

### IV. Uses

The methods and compositions of the disclosure for generating human induced mesenchymal stem cells, and progenitors thereof, allow for efficient and robust availability of these cell populations for a variety of uses. For example, the iMSCs generated using the methods and compositions can be used in the study of human mesenchymal cell development and differentiation, including differentiation to various other cell types. The iMSCs generated by the method of the disclosure thus also serve as a source for stem cells for the generation of a variety of more differentiated cell types that have uses both for research purposes and clinically.

The iMSCs generated by the disclosed methods can be cultured in vitro and used as a source of for obtaining other agents, such as a source of immunomodulatory agents (e.g., IL-10) that can be secreted by the iMSCs, or a source of extracellular vesicles (EV), such as exosomes, that are generated by the iMSCs. Such agents can be purified from the iMSCs, or culture media in which the cells are grown, by standard methods known in the art.

Additionally, the iMSCs generated by the disclosed methods can be used for therapeutic purposes, for example in clinical situations in which mesenchymal stem cells derived by other methods have been used or are being contemplated or tested for use. The iMSCs used for therapeutic purposes may be unmodified cells or may be cells that have been modified (e.g., to express a therapeutic agent, such as a recombinant secreted protein or an mRNA agent). Non-limiting examples of clinical situations in which mesenchymal stem cells have been used and continue to be tested include cardiac injuries (e.g., ischemic cardiomyopathy) and autoimmune diseases (e.g., Crohn's disease, multiple sclerosis, systemic lupus erythematosus, systemic sclerosis).

### V. Compositions

In other aspects, the disclosure provides compositions related to the methods of generating human induced mesenchymal stem cells, including culture media and kits.

In one aspect, the disclosure provides a two-stage culture media for obtaining human mesenchymal stem cell progenitor cells, the two-stage culture media comprising (i) a first stage media comprising a WNT pathway agonist and a BET pathway antagonist; and (ii) a second stage media comprising a PDGF pathway agonist, an IGF1 pathway agonist and an FGF-beta pathway agonist. Non-limiting examples of suitable agents, and concentrations therefor, include those described in subsection II above. In an embodiment, the first stage media comprises CHIR98014 (e.g., at 0.5 uM) and (+)-JQ1 (e.g., at 50 nM) and the second stage media comprises PDGF-BB (e.g., at 10 ng/ml), IGF1 (e.g., at 20 ng/ml) and FGF-beta (e.g., at 10 ng/ml). The first and second stage media can comprise a base media, such as those described in sections II and III above (e.g., DMEM/F12 with 10% FBS).

In another aspect, the disclosure provides a kit for generating human induced mesenchymal stem cells, the kit comprising the two-stage culture media described above and a sample of human pluripotent stem cells (e.g., hESC line or iPSC line), along with instructions for culturing the human pluripotent stem cells using the two-stage culture media to generate iMSCs (e.g., according to the protocol described herein).

The present invention is further illustrated by the following examples, which should not be construed as further limiting. The contents of figures and all references, patents and published patent applications cited throughout this application are expressly incorporated herein by reference.

### EXAMPLES

### Example 1: Culture Protocol for the Generation of Induced Mesenchymal Stem Cells

A three-stage protocol for the generation of induced mesenchymal stem cells (iMSCs) from human embryonic stem cells (hESCs) was developed. The protocol is illustrated schematically in FIG. 1. The protocol first involves a 4-day induction stage, using an induction medium, followed by a 10-day enhancement stage, using an enhancement medium. These first two stages provide a culture of human iMSC progenitors capable of further differentiation to mature iMSCs. Stage three thus involves further differentiation of the iMSC progenitors to mature iMSCs by further culture in maintenance medium for an additional period of time (e.g., at least one to two weeks).

To maintain hESCs in culture prior to initiation of the iMSC protocol, hESCs were grown on 6-well plates coated with 1% Matrigel (Corning, Cat. #354277) in mTeSR1 culture medium (STEMCELL Technologies, Cat. #85850). The culture medium was replaced fresh each day. When cells reached 80-90% confluence, the culture medium was aspirated and 1 mL/well of Versene (Thermo Fisher, Cat. #15040066) was added. Culture plates were incubated at 37° C. for 6-7 minutes, the Versene was gently aspirated and the cells were resuspended in mTeSR1 medium supplemented with 5 uM Y-27632 (Tocris, Cat. #1254). Cells were counted and seeded at 100k/well live cells on new plates. Cells were maintained in mTeSR1 medium until use in the iMSC protocol, with the culture medium changed daily.

### Stage 1 Induction Protocol

For the first stage of the iMSC protocol, on day −1 (one day before the start of the differentiation protocol), 60-100k/well live hESCs were seeded onto 6-well plates coated with 1% Geltrex (Thermo Fisher, Cat. #1413301) (approx. 8000 cells/cm²), using 2 mL/well of mTeSR1 culture medium supplemented with 5 uM Y-27632. This passage was marked as passage 0 (P0). Then, on day 0 (the start day of differentiation), the medium was changed to 2 mL/well of Induction Medium. The Induction Medium recipe was: DMEM/F12 (Thermo Fisher, Cat. #11320033)+10% fetal bovine serum (FBS; Thermo Fisher, Cat. #10082147) supplemented with 50 nM (+)-JQ1 (Sigma Aldrich, Cat. #SML1524) and 0.5 uM Chir98014 (Tocris, Cat. #6695). (+)-JQ1 is a BET (bromodomain and extra-terminal motif) family inhibitor (antagonist). Chir98014 is an activator (agonist) of the WNT pathway (through GSK inhibition). Cells were maintained in Induction Medium for two days (day 0-day 2), followed by a culture medium change and further culture in fresh Induction Medium for two more days (day 2-day 4), for a total of four days of culture in Induction Medium for stage 1.

### Stage 2 Enhancement Protocol

On day 4, the Induction Medium was aspirated from the cultures and 2 mL/well of Enhance Medium was added. The Enhance Medium recipe was: DMEM/F12 media+10% FBS supplemented with 10 ng/mL PDGF-BB (R&D Systems, Cat. #220-BB), 20 ng/mL IGF1 (R&D Systems, Cat. #291-G1) and 10 ng/mL FGF-beta (R&D Systems, Cat. #3718-FB). Cells were maintained in Enhance Medium from day 4 to day 14, with medium changes every two days.

When cells reached 90% confluence in Enhance Medium, typically between day 6 to day 8 (depending on the initial seeding density), the cells were passaged to P1. For passaging, the Enhance Medium was aspirated and cells were washed once with phosphate buffered saline (PBS). Cells were treated with 1 mL/well Accutase (Thermo Fisher, Cat. #A1110501) and incubated in a 37° C. incubator for 5-6 minutes. DMEM/F12 media with 10% FBS was added to stop cell dissociation. Cells were suspended, transferred into a centrifuge tube and spun down at 300 g for three minutes. After centrifugation, the supernatant was discarded and the cell pellet was resuspended in Enhance Medium supplemented with 5 uM Y-27632. Cells were seeded at about 150k/well live cells onto new 6-well plates coated with 1% Geltrex (approx. 15,000 cells/cm²). Cells were maintained in Enhance Medium to day 14, with fresh medium changes every two days. If cells reached 90% confluence again before day 14, cells were passaged again in Enhance Medium as described above.

### Stage 3 Maintenance Protocol

On day 14, the culture medium was changed to Maintenance Medium, which was then changed every four days. The Maintenance Medium recipe was: DMEM/F12+10% FBS, supplemented with 2 mM GlutaMax (Thermo Fisher, Cat. #35050-061). When cells reached 90% confluence, cells were passaged.

If cells were not passaged to P2 during the enhancement stage (i.e., before day 14), then P2 passage was carried out on day 14 with Maintenance Medium as follows. Cell culture medium was aspirated and cells were washed once with PBS. Accutase (1 mL/well) was added and cells were incubated in a 37° C. incubator for 5-6 minutes. DMEM/F12+10% FBS was added to stop the dissociation and suspended cells were transferred to a centrifuge tube. Cells were centrifuged at 300 g for three minutes, the supernatant was discarded and the cell pellet was resuspended in Maintenance Medium supplemented with 5 uM Y-27632. Cells were transferred from wells to 10 cm dishes (approx. 10,000 cells/cm²) coated with 0.1% Gelatin (Millipore, Cat. #ES-006). Additional medium was added to reach a total volume of 8-10 mL/dish.

Cells were then maintained in Maintenance Medium (with changes every four days) and each time cell density reached 90% confluence, the cells were split to the next passage as follows. Cell culture medium was aspirated and cells were washed once with PBS. TrypLE Select (5 mL/dish) was added and cells were incubated in a 37° C. incubator for 8-10 minutes. DMEM/F12+10% FBS was added to stop the dissociation and suspended cells were transferred to a centrifuge tube. Cells were centrifuged at 300 g for three minutes, the supernatant was discarded and the cell pellet was resuspended in Maintenance Medium supplemented with 5 uM Y-27632. Cells were seeded at 300k/dish live cells on 10 cm dishes (approx. 5,000 cells/cm²) coated with 0.1% Gelatin. Additional medium was added to reach a total volume of 8-10 mL/dish. Mesenchymal stem cell-like cells started to appear from day 21 of culture.

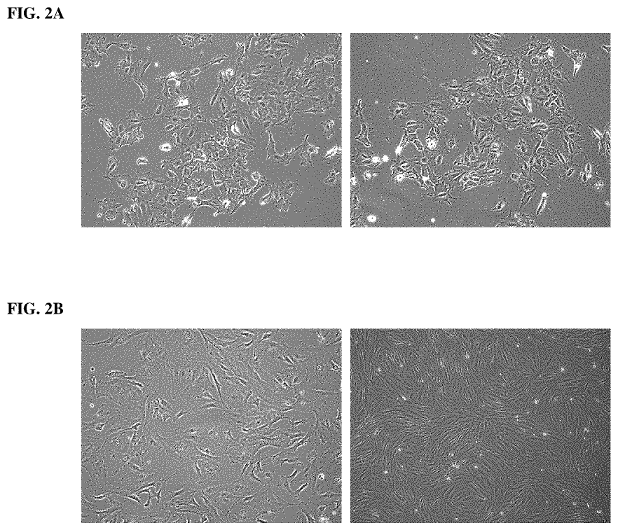

Cell morphology was examined microscopically at different stages of the differentiation protocol, the results of which are shown in FIG. 2A-B. Cells from day 4 to day 14 were at the mesodermal stage, representative examples of which are shown in FIG. 2A (left and right panels). Cells at this stage displayed nascent mesodermal-like morphology with a relatively large nucleus, with some cells having filopodia. From day 21 onward, cells started to acquire mesenchymal/fibroblast-like morphology, as shown in FIG. 2B (left panel)(low density 10×). At higher density, cells became spindle-like in shape, and a wavy pattern could be observed, as shown in FIG. 2B (right panel)(high density 5×).

The induced MSCs maintained a constant proliferation rate in culture for at least three months. The cells were expanded for validation tests, described further below.

### Example 2: Surface Marker Expression of Induced Mesenchymal Stem Cells

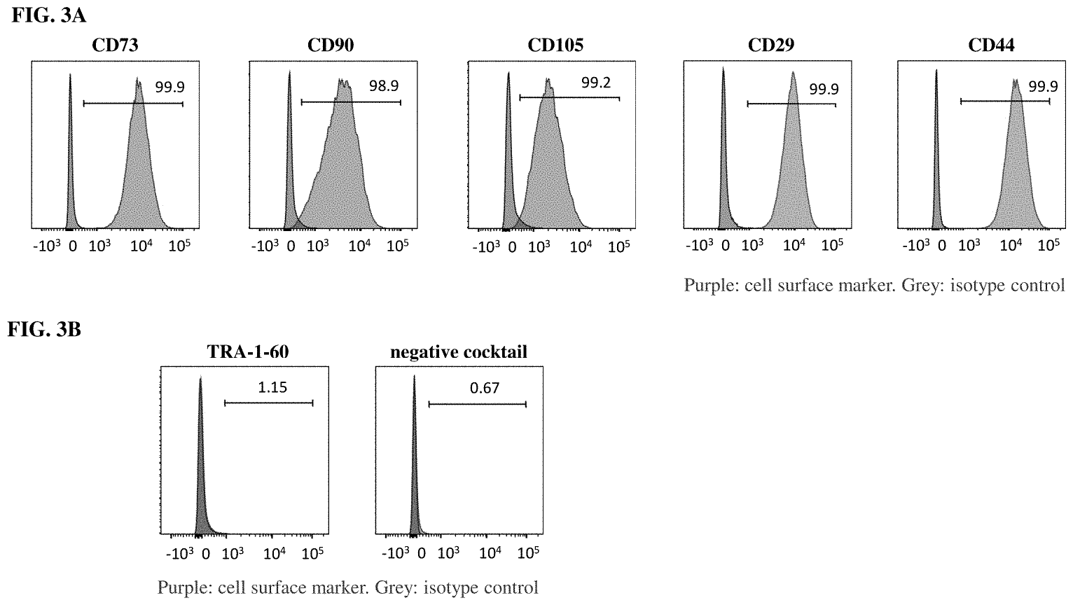

Surface marker expression of the induced mesenchymal stem cells (iMSCs) was examined by standard FACS analysis. Positive markers for MSCs examined were CD73, CD90, CD105, CD29 and CD44. As shown in FIG. 3A, the iMSCs generated by the differentiation protocol described in Example 1 exhibited surface expression of each of these mesenchymal markers. Negative markers examined included TRA-1-60, CD11b, CD19, CD34, CD45 and HLA-DR. As shown in FIG. 3B, the iMSCs did not express any of the negative markers tested (wherein the “negative cocktail” contained anti-CD11b, anti-CD19, anti-CD34, anti-CD45 and anti-HLA-DR antibodies).

### Example 3: Trilineage Differentiation of Induced Mesenchymal Stem Cells

To examine the differentiation ability of the iMSCs generated according to the protocol described in Example 1, cells were further treated using protocols for either adipocyte, osteocyte or chondrocyte differentiation.

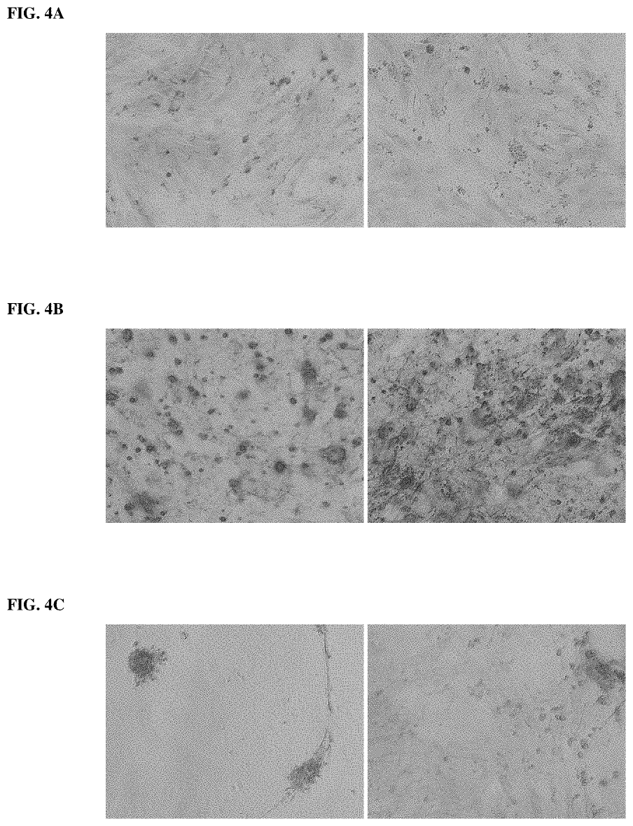

For adipogenesis, iMSCs were cultured using Adipogenesis Differentiation Kit (Thermo Fisher, Cat. #A1007001) according to the manufacturer's instructions. Following 21 days of adipocyte differentiation, cells were stained by Oil Red solution (Sigma Aldrich, Cat. #01391), which stains the lipid particles in adipocytes red. Results are shown in FIG. 4A, showing the ability of the iMSCs to differentiate into adipocytes.

For osteogenesis, iMSCs were cultured using Osteogenesis Differentiation Kit (Thermo Fisher, Cat. #A1007201) according to the manufacturer's instructions. Following 14 days of osteocyte differentiation, cells were stained by Alizarin Red solution (Sigma Aldrich, Cat. #TMS-008-C), which stains osteocytes red. Results are shown in FIG. 4B, showing the ability of the iMSCs to differentiate into osteocytes.

For chondrogenesis, iMSCs were cultured using Chondrogenesis Differentiation Kit (Thermo Fisher, Cat. #A1007101) according to the manufacturer's instructions. Following 14 days of chondrocyte differentiation, cells were stained by Alcian Blue solution (Sigma Aldrich, Cat. #B8438, which stains chondrocytes blue. Results are shown in FIG. 4C, showing the ability of the iMSCs to differentiate into chondrocytes.

Overall, the results of FIG. 4A-C demonstrate the ability of the iMSCs for trilineage differentiation.

### Example 4: Immunomodulation of Induced Mesenchymal Stem Cells

In this example, various studies were conducted to examine the immunomodulatory capacity of the iMSCs generated according to the differentiation protocol described in Example 1.

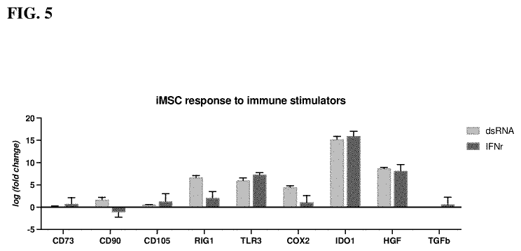

In a first set of studies, iMSCs were treated with immune stimulators, either double-strand RNA (dsRNA) or recombinant human interferon gamma (IFNr)(R&D Systems, Cat. #35-IF-100). iMSCs (100k cells/sample) were cultured with dsRNA (500 ng/sample) or IFNr (10 ng/ml) for 24 hours. After treatment, the immunomodulatory capability of the iMSCs was analyzed by quantitative real-time PCR of marker genes potentially upregulated by the immune stimulators tested. Immunostimulatory marker genes examined were: RIG1, TLR3, COX2, IDO1, HGF and TGF-beta. Control genes (for MSC surface markers) examined were: CD73, CD90 and CD105. The results are shown in FIG. 5. The results demonstrate that treatment of the iMSCs with immune stimulators led to increased expression of multiple genes associated with immunostimulation (particularly IDO1, TLR3 and HGF), whereas the control marker genes were not upregulated.

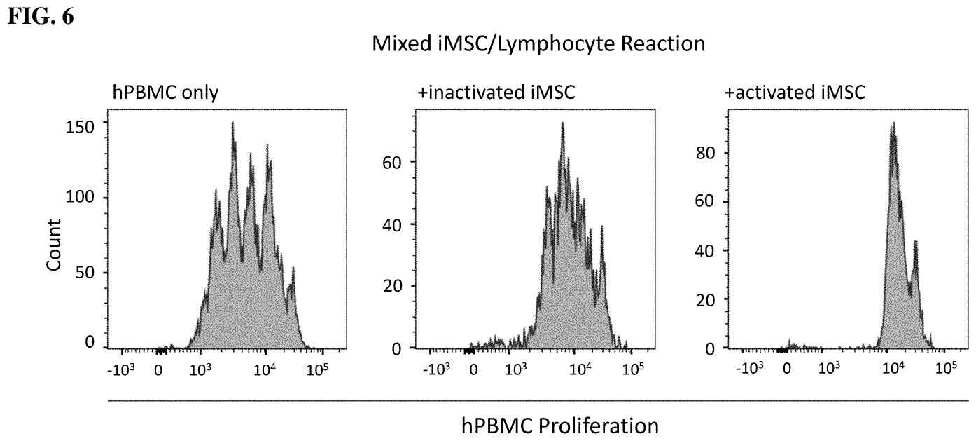

In a second set of experiments, the iMSC were used in mixed lymphocyte reactions (MLR). Human peripheral blood mononuclear cells (hPBMC)(Lonza, Cat. #cc-2702) were labeled with CellTrack Violet (Thermo Fisher, Cat. #C34557) to indicate cell proliferation. iMSC (50k cells/well of a 24 well plate; approx. 25,000 cells/cm²) were pre-treated with 10 ng/ml of IFNr for three days, referred to as “activated iMSC”, whereas iMSC without IFNr pre-treatment are referred to as “inactivated iMSC.” For the MLR, hPBMC were added on top of iMSC and the cells were co-cultured for three days in a co-culture media supplemented with recombinant human IL-2 (R&D Systems, Cat. #202-IL) and anti-CD3 (eBioscience, Cat. #16-0037-81) to promote T cell proliferation. After three days, hPBMC were collected and analyzed by flow cytometry. As shown in FIG. 6, five peaks were observed in the “hPBMC only” group, indicating the cells proliferated four times. The indistinguishable peaks in the “+inactivated iMSC” group suggests that proliferation of hPBMC was interfered with by co-culture with the inactivated iMSC. The single dominant peak in the “+activated iMSC” group indicated that cell proliferation happened just once and then was halted during co-culture with the activated iMSC.

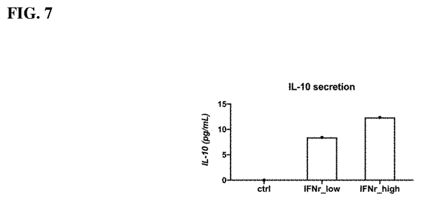

In a third set of experiments, the production of the anti-inflammatory factor IL-10 by the iMSCs was examined after treatment with IFNr. iMSCs (50k cells/well of a 24 well plate; approx. 25,000 cells/cm²) were treated with low concentration (20 ng/mL) or high concentration (50 ng/mL) IFNr for three days to stimulate secretion of IL-10. The control group was the same culture conditions but without IFNr treatment. After three days, media supernatant was collected and the level of IL-10 secretion was quantified by standard ELISA analysis (human IL-10 Quantikine ELISA kit, R&D Systems, Cat. #D1000B). As shown in FIG. 7, secretion of IL-10 by iMSCs was dose-dependent to the concentration of IFNr treatment.

### Example 5: Extracellular Vesicle Production by Induced Mesenchymal Stem Cells

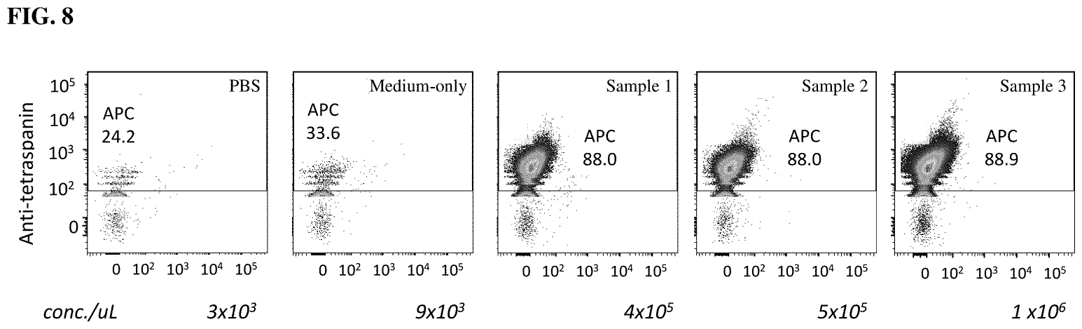

In this example, extracellular vesicle production by the iMSCs was quantified. Cells first were washed with PBS and then cultured in Maintenance Medium (described in Example 1) containing exosome-depleted FBS. After four days of culturing, medium was collected and the total volume was measured. The iMSCs were dissociated from the culture vesicles and cell numbers were counted. Extracellular vesicles were quantified using a FACS-based analysis that targeted Tetraspanin protein on the vesicle membrane. The results for three representative samples are shown in FIG. 8 (with PBS and medium-only controls), demonstrating production of extracellular vesicles by the iMSCs.

### EQUIVALENTS

Those skilled in the art will recognize, or be able to ascertain using no more than routine experimentation, many equivalents of the specific embodiments of the invention described herein. Such equivalents are intended to be encompassed by the following claims

1. A method of generating human mesenchymal stem cell progenitor cells comprising:
(a) culturing human pluripotent stem cells in a culture media comprising a WNT pathway agonist and a BET pathway antagonist for at least two days to generate induced cells; and
(b) culturing the induced cells from step (a) in a culture media comprising a PDGF pathway agonist, an IGF1 pathway agonist and an FGF-beta pathway agonist for at least ten days to generate human mesenchymal stem cell progenitor cells.

2. The method of claim 1, wherein step (a) comprises culturing the human pluripotent stem cells in the culture media comprising a WNT pathway agonist and a BET pathway antagonist on day 0 to day 4 to generate induced cells.

3. The method of claim 2, wherein step (b) comprises culturing the induced cells from step (a) in a culture media comprising a PDGF pathway agonist, an IGF1 pathway agonist and an FGF-beta pathway agonist on day 4 to day 14 to generate human mesenchymal stem cell progenitor cells.

4. The method of claim 1, wherein the human pluripotent stem cells are human embryonic stem cells.

5. The method of claim 1, wherein the WNT pathway agonist is a glycogen synthase kinase 3 (Gsk3) inhibitor.

6. The method of claim 1, wherein the WNT pathway agonist is CHIR98014.

7. The method of claim 1, wherein the WNT pathway agonist is selected from the group consisting of CHIR98014, CHIR99021, SB 216763, SB 415286, LY2090314, 3F8, A 1070722, AR-A 014418, BIO, AZD1080, WNT3A, and combinations thereof.

8. The method of claim 1, wherein the WNT pathway agonist is present in the culture media at a concentration within a range of 0.25-0.75 μM.

9. The method of claim 1, wherein the WNT pathway agonist is present in the culture media at a concentration of 0.5 μM.

10. The method of claim 9, wherein the WNT pathway agonist is CHIR98014 at a concentration of 0.5 μM.

11. The method of any one of claim 1, wherein the BET pathway antagonist is a triazolo-diazepine compound.

12. The method of claim 11, wherein the triazolo-diazepine compound is (+)-JQ1.

13. The method of claim 1, wherein the BET pathway antagonist is selected from the group consisting of (+)-JQ1, TEN-010, OTX015, I-BET762, I-BET151, BAY1238097, ABBV-744, ABBV-075, iBET-BD1, iBET-BD2, SJ432, RVX-208, MS417, AZD5153, and combinations thereof.

14. The method of claim 13, wherein the BET pathway antagonist is present in the culture media at a concentration within a range of 25-75 nM.

15. The method of claim 14, wherein the BET pathway antagonist is (+)-JQ1 at a concentration of 50 nM.

16. The method of claim 1, wherein the PDGF pathway agonist is PDGF-BB.

17. The method of claim 16, wherein PDGF-BB is present in the culture media at a concentration within a range of 7.5-12.5 ng/ml.

18. The method of claim 16, wherein PDGF-BB is present in the culture media at a concentration of 10 ng/ml.

19. The method of claim 1, wherein the IGF1 pathway agonist is IGF1.

20. The method of claim 19, wherein IGF1 is present in the culture media at a concentration within a range of 15-25 ng/ml.

21. The method of claim 19, wherein IGF1 is present in the culture media at a concentration of 20 ng/ml.

22. The method of claim 1, wherein the FGF-beta pathway agonist is FGF-beta.

23. The method of claim 22, wherein FGF-beta is present in the culture media at a concentration within a range of 7.5-12.5 ng/ml.

24. The method of claim 22, wherein FGF-beta is present in the culture media at a concentration of 10 ng/ml.

25. The method of claim 1, wherein the culture media in step (a) and step (b) comprises a base media comprising serum.

26. The method of claim 25, wherein the base media comprising serum is DMEM/F12 media with 10-15% fetal bovine serum.

27. The method of claim 1, which further comprises culturing the human mesenchymal stem cell progenitor cells for at least 7 days in a culture medium comprising a base media comprising an L-glutamine supplement.

28. The method of claim 27, wherein the L-glutamine supplement is an L-alanine-L-glutamine dipeptide.

29. The method of claim 27, wherein the base media comprising an L-glutamine supplement is DMEM/F12 media with 10% fetal bovine serum supplemented with GlutaMAX™.

30. A method of generating human induced mesenchymal stem cells (iMSC) comprising:
(a) culturing human pluripotent stem cells in a culture media comprising a WNT pathway agonist and a BET pathway antagonist for at least two days to generate induced cells;
(b) culturing the induced cells from step (a) in a culture media comprising a PDGF pathway agonist, an IGF1 pathway agonist and an FGF-beta pathway agonist for at least 10 days to generate human mesenchymal stem cell progenitor cells; and
(c) culturing the human mesenchymal stem cell progenitor cells from step (b) in a culture media comprising a base media and an L-glutamine supplement for at least 7 days to generate iMSCs.

31. The method of claim 30, wherein step (a) comprises culturing the human pluripotent stem cells in the culture media comprising a WNT pathway agonist and a BET pathway antagonist on day 0 to day 4 to generate induced cells.

32. The method of claim 31, wherein step (b) comprises culturing the induced cells from step (a) in a culture media comprising a PDGF pathway agonist, an IGF1 pathway agonist and an FGF-beta pathway agonist on day 4 to day 14 to generate human mesenchymal stem cell progenitor cells.

33. The method of claim 30, wherein step (c) comprises culturing the human mesenchymal stem cell progenitor cells for at least 14 days in the base media and L-glutamine supplement.

34. The method of claim 30, wherein the human pluripotent stem cells are human embryonic stem cells.

35. The method of claim 30, wherein the iMSCs express one or more surface markers selected from the group consisting of CD73, CD90, CD105, CD29, CD44, and combinations thereof.

36. The method of claim 30, wherein the iMSCs are capable of further differentiation into adipocytes, osteocytes or chondrocytes.

37. The method of claim 30, wherein the WNT pathway agonist is a glycogen synthase kinase 3 (Gsk3) inhibitor.

38. The method of claim 37, wherein the WNT pathway agonist is CHIR98014.

39. The method of claim 30, wherein the WNT pathway agonist is selected from the group consisting of CHIR98014, CHIR99021, SB 216763, SB 415286, LY2090314, 3F8, A 1070722, AR-A 014418, BIO, AZD1080, WNT3A, and combinations thereof.

40. The method of claim 39, wherein the WNT pathway agonist is present in the culture media at a concentration within a range of 0.25-0.75 μM.

41. The method of claim 40, wherein the WNT pathway agonist is present in the culture media at a concentration of 0.5 μM.

42. The method of claim 41, wherein the WNT pathway agonist is CHIR98014 at a concentration of 0.5 μM.

43. The method of claim 30, wherein the BET pathway antagonist is a triazolo-diazepine compound.

44. The method of claim 43, wherein the triazolo-diazepine compound is (+)-JQ1.

45. The method of claim 30, wherein the BET pathway antagonist is selected from the group consisting of (+)-JQ1, TEN-010, OTX015, I-BET762, I-BET151, BAY1238097, ABBV-744, ABBV-075, iBET-BD1, iBET-BD2, SJ432, RVX-208, MS417, AZD5153, and combinations thereof.

46. The method of claim 45, wherein the BET pathway antagonist is present in the culture media at a concentration within a range of 25-75 nM.

47. The method of claim 46, wherein the BET pathway antagonist is (+)-JQ1 at a concentration of 50 nM.

48. The method of claim 30, wherein the PDGF pathway agonist is PDGF-BB.

49. The method of claim 48, wherein PDGF-BB is present in the culture media at a concentration within a range of 7.5-12.5 ng/ml.

50. The method of claim 48, wherein PDGF-BB is present in the culture media at a concentration of 10 ng/ml.

51. The method of claim 30, wherein the IGF1 pathway agonist is IGF1.

52. The method of claim 51, wherein IGF1 is present in the culture media at a concentration within a range of 15-25 ng/ml.

53. The method of claim 51, wherein IGF1 is present in the culture media at a concentration of 20 ng/ml.

54. The method of claim 30, wherein the FGF-beta pathway agonist is FGF-beta.

55. The method of claim 54, wherein FGF-beta is present in the culture media at a concentration within a range of 7.5-12.5 ng/ml.

56. The method of claim 54, wherein FGF-beta is present in the culture media at a concentration of 10 ng/ml.

57. The method of claim 30, wherein the culture media in steps (a), (b) and (c) comprises a base media comprising serum.

58. The method of claim 57, wherein the base media comprising serum is DMEM/F12 media with 10-15% fetal bovine serum.

59. The method of claim 30, wherein the L-glutamine supplement is an L-alanine-L-glutamine dipeptide.

60. The method of claim 59, wherein the base media comprising an L-glutamine supplement in step (c) is DMEM/F12 media with 10% fetal bovine serum supplemented with GlutaMAX™.

61. The method of claim 30, which further comprises isolating from the culture extracellular vesicles generated by the iMSCs.

62. A two-stage culture media for obtaining human mesenchymal stem cell progenitor cells, the two-stage culture media comprising (i) a first stage media comprising a WNT pathway agonist and a BET pathway antagonist; and (ii) a second stage media comprising a PDGF pathway agonist, an IGF1 pathway agonist and an FGF-beta pathway agonist.
